(19) United States
(12) Patent Application Publication
(10) Pub. No.: US 2005/0045614 A1
(43) Pub. Date: Mar. 3, 2005

(54) Tissue irradiation system and apparatus

(76) Inventors: Mark Hayzlett, Bridgewater, NJ (US); Michael J. Honey, Stirling, NJ (US)

Correspondence Address: FISH & RICHARDSON P.C., CITIGROUP CENTER 52ND FLOOR, 153 EAST 53RD STREET, NEW YORK, NY, 10022-4611, US

(21) Appl. No.: 10/929,231

(22) Filed: Aug. 30, 2004

Related U.S. Application Data
(60) Provisional application No. 60/498,941, filed on Aug. 29, 2003.

(51) Int. Cl. A61L 2/08; H05B 3/00

(52) U.S. Cl. 219/217; 422/22; 435/1.1

(57) ABSTRACT

Systems and techniques are described for constructing a gamma container. In general, the techniques include sandwiching a material between layers of cooling pads that conduct heat energy laterally away from the material being irradiated to a cold sink.

This application claims priority of U.S. Provisional Application No. 60/498,941, filed Aug. 29, 2003, the disclosure of which is hereby incorporated herein by reference in its entirety.

### TECHNICAL FIELD

The following description relates to systems and techniques for conducting energy from materials.

### BACKGROUND

Gamma radiation transfers energy to material primarily by scattering, which involves elastic collisions between incident photons and unbound (or weakly bound) electrons in which the incident energy is shared between the scattered electron and the deflected photon. These electrons recoil a short distance as unbound electrons, giving up energy to the molecular structure of the material as they collide with other electrons, causing ionization and free-radical formation. The scattered gamma ray carries the balance of the energy as it moves off through the material, possibly to interact again with another atomic electron. Gamma rays typically penetrate relatively deeply into the tissue before scattering occurs. Gamma radiation typically requires a low dose rate in combination with a high exposure period.

The physical or physiological properties of active compounds may be altered by variations in the compounds' surrounding environment. For example, changes in pH, ionic strength, or temperature can result in reversible or irreversible changes in the character of compounds.

Radiation sterilization is widely used in industry for a variety of products and both dosage levels and its biological effects are well known. Gamma sterilization can be effective in killing microbial organisms. Gamma radiation sufficient to effectively kill microorganisms also may alter the structure of biological and other compounds.

### SUMMARY OF THE DISCLOSURE

The invention relates to systems, apparatus and methods for conducting energy away from materials.

Gamma radiation is often utilized to inactivate undesirable organisms, so that growth of the undesirable organisms is minimized or eliminated.

Inactivation of undesirable organisms from allograft tissue may be accomplished by using gamma rays from Cobalt 60. A sufficient accumulated absorbed dose level to reduce or eliminate a given spectrum of these organisms may also have a damaging effect upon the structure and biochemical nature of unprotected tissue. An aspect of the invention is to provide a system that will allow gamma rays to have the desired effect upon micro-organisms while protecting allograft or other tissue from degradation.

The invention includes an apparatus into which soft human or animal tissue is placed between layers of energy conductive medium that provides support and thermal management during exposure to gamma irradiation at temperatures below −20° Celcius. The apparatus provides a static mechanism to drain pure energy effects absorbed by the tissue(s). Energy is withdrawn in a controlled direction to a cold sink at the side of the apparatus without significant attenuation that would interfere with the microbe-killing effects of gamma rays penetrating the front of the apparatus. The rate of energy loss through the conductive layers results in a diminished level of energy available for degenerative biochemical reactions that would otherwise occur within the tissue during gamma exposure.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a section through loaded container according to one aspect.

FIG. 2 is an example of a cooling pad layer of FIG. 1.

FIG. 3 is an example of a biological layer of FIG. 1.

FIG. 4 is an example of a hinge arrangement for the container.

Like reference symbols in the various drawings indicate like elements.

### DETAILED DESCRIPTION

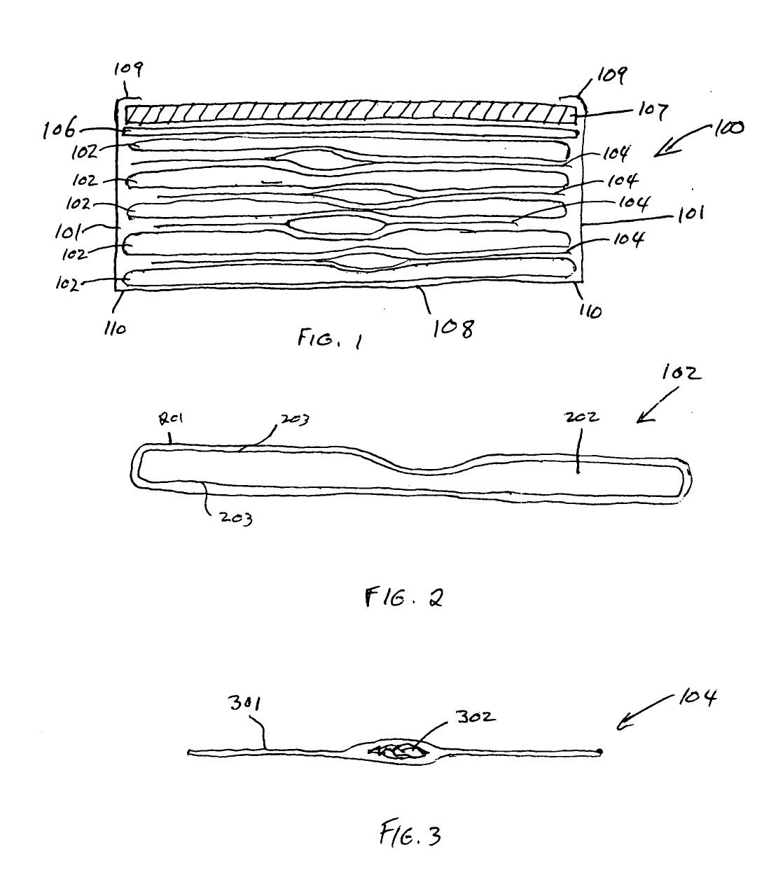

FIG. 1 illustrates an example of an apparatus useful for exposing tissue to gamma rays while simultaneously protecting the tissue.

As shown in FIG. 1, a container 100 has a bottom 108 and upstanding sides 101 forming right angles with the bottom 108, at edges 110. Bottom 108 can be rectangular or square in shape. As will be appreciated, there are also upstanding sides along at least one of the other edges (not shown) of bottom 108. In the embodiment illustrated, three of the sides 101 are fixed in relation to bottom 108. A fourth side 101 can be hingedly connected to the two sides 101 that are adjacent the fourth side 101. If fourth side 101 is hingedly attached, it can be rotated between an open orientation that is approximately parallel to bottom 108, and a closed orientation approximately perpendicular to bottom 108. Hinge connections on the sides 101 that are adjacent to fourth side 101, and near bottom 108, could be used. As will be readily appreciated, these connections can be rotatable rivets, screws or bolts 118 which hold flanges 116 that are on either side of fourth side 101 to each of the two adjacent sides 101. (See FIG. 4.) The fourth side 101 can rotate as shown by arrow A in FIG. 4.

As shown in FIG. 1, at least two of the sides 101 have a retaining flange 109 along the top edges of sides 101. Each such retaining flange extends out approximately perpendicularly to sides 101 (and therefore approximately parallel to bottom 108).

As shown in the FIG. 1 example, when in use, container 100 can be loaded with a plurality of alternating layers 102, 104, 102, 104, 102, 104, 102, 104, 102. For simplicity of illustration, five layers 102 and four layers 104 are shown, but either more or fewer layers can, of course, be used depending on the height of sides 101, the thickness of the layers 102, 104, the tissue being gamma irradiated, etc. For example, four layers 102 alternating with three layers 104 can be used.

FIG. 2 illustrates an example of cooling pad layer 102. Each cooling pad layer 102 comprises a support 202 whose surface can be coated with a heat conduction material 203. Support 202 and heat conduction material 203 are sealed in a sleeve 201.

Support 202 comprises a hydrophilic substance, such as open-cell polyurethane foam, fiberglass mesh, leather, felt or terrycloth. Heat conduction material 203 is coated on the outer surfaces of support 202. Suitable heat conduction materials 203 include boron nitride powder and beryllium copper.

In preparing cooling pad layer 102, a heat conduction material 203 forms a coating on a dry support 202 and the coated support is put into a sleeve 201. A small amount of water is added to the sleeve (say 100-150 mls.). All the water added should be absorbed by the coated support and the support should not be fully saturated. Almost all the air is then removed from sleeve 201 and the sleeve is then sealed to be airtight such as by heat-sealing. Sleeve 201 can be any suitable plastic, such as 3-mil thick propylene, high density polyethylene or mylar.

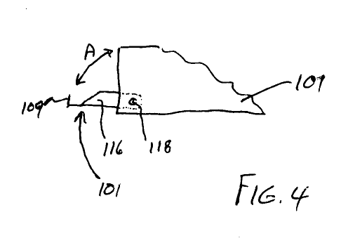

FIG. 3 illustrates an example of biological layer 104. Each layer 104 comprises tissue 302 and sleeve 301. Tissue 302 can be soft human or animal tissue, such as tendon or human sheet dermis or any material which is to be subjected to gamma radiation, such as a synthesized polymeric. Sleeve 301 can be a polypropylene bag, high density polyethylene or mylar. Sleeve 301 is sealed after tissue 302 is placed therein and almost all air has been removed from sleeve 301. Tissue 302 can be wetted tissue.

Referring back to FIG. 1, retention layer 106 can be slightly flexible so that it can be flexed and its edges then inserted under retaining flanges 109. Upon release, retention layer 106 can resume a more planar configuration and its edges can extend beyond the width of the opening formed by retaining flanges 109 in the top of container 100. Retention layer 106 can comprise a stainless steel screen, for example. Retention layer 106 is useful to keep alternating layers 102, 104, 102, etc. in place prior to closing container 100. Retention layer 106 also can provide a smooth surface along which cover layer 107 can slide.

Cover layer 107 is relatively stiff and inflexible compared to retention layer 106 and can slide along the top of retention layer 106, below retaining flanges 109 which are on sides 101. Once container 100 is loaded with alternating layers 102, 104, 102, etc., and once retention layer 106 is in place, cover layer 107 is slid into place above retention layer 106. It is sized so that it closes the top of container 100 once cover layer 107 is in place. Now, fully loaded, container 100 is further closed by moving fourth side 101 to its closed position, perpendicular to bottom 108.

Cover layer 107 can be made of high-density polyethylene (HDPE) or any suitable rigid plastic material.

It should be noted that all three sides 101 and fourth side 101 can have retaining flanges 109 so that all four edges of cover layer 107 will be retained thereby when fourth side 101 is in its closed position.

In another example, fourth side 101 is not hingedly connected to the two adjacent sides 101, but can be completely removable from container 100. In this example, after cover layer 107 has been slid into place, fourth side 101 can be placed on container 100. In this example, retaining flange 109 can have an opposing flange 109, arranged so that one retaining flange 109 frictionally fits over cover layer 107 and the opposing retaining flange 109 frictionally fits the outside of bottom 108 of container 100.

When container 100 is loaded and is in use, cover layer 107 faces toward a gamma ray source, such as Cobalt 60. The gamma rays can penetrate through cover layer 107, and the other layers, namely, retention layer 106 and alternating layers 102, 104, 102, etc. and finally through the bottom layer 108 of container 100. Before the tissue to be exposed to gamma irradiation is treated, the loaded container 100 is cooled to −60 to −80° C. and maintained at that temperature. When container 100 is rectangular, container 100 is positioned with one of the two shorter sides 101 down and the other short side up, and dry ice is placed along and in contact with both longer sides 01, which are vertically arranged in this example. The alternating layers 102, 104, 102, etc. are prepared as described above and are firmly held in contact with one another in container 100. It is important that a cooling pad layer 102 be in contact with each side of each biological layer 104 in loaded container 100. Cooling pad layers 102 can, and should, compress somewhat around the tissue in biological layer 104 to maintain a snug fit as container 100 is being loaded.

Bottom 108, sides 101 and fourth side 101 can all be 0.033 in. thick aluminum. Alternatively, a 0.005 in. thick formed aluminum food tray has been found to be a useful substitute for the 0.033 in. thick aluminum.

In one example, a rectangular container having bottom and sides made of 0.033 inches thick aluminum is used. Each of five cooling pad layers is made of hydrophilic polyurethane open-cell foam sheet, roughly 0.2 inches thick and 6.5 by 9.5 inches in plan view. Each side of each cooling pad is saturated with boron-nitride powder (from Saint-Gobain, CTL40, 3 to 5 grams) while dry, then is placed within a 3 mil thick heat-sealed polypropylene sleeve. Before final vacuum sealing, the bag is filled with approximately 100 ml of RO-purified or demineralized water. Sufficient air is withdrawn from the cooling pad at final sealing to remove most excess air from between the polyurethane foam pad and the polypropylene sleeve. The tissue used in each of the four biological layers is tendon. The retention layer and the cover layer are added and the fourth side is closed. The loaded container with the tendon layers present can be regarded as a single conducting unit. The calculated heat conductance coefficient (k) for this unit is determined to be 80 W/m−K from one end of the unit to the other. When the container is fully loaded and closed, the five cooling layers are in firm contact with and support the four adjacent biological layers. The loaded container is chilled to about −60 to −80° C. The container is placed with one of its short sides down, with its plastic cover layer facing the gamma ray source.

The gamma ray source can release radiation at a controlled rate between about 2 and about 10 kilo-Grays/hour. Tissue can typically absorb about 10 to about 40 kilo-Grays of energy with little or no energy-caused degradation.

In use, the container is kept at about −60 to −80° C. by contacting both long sides of the container with a cold sink, such as dry ice, while being exposed to gamma radiation.

A number of embodiments of the invention have been described. Nevertheless, it will be understood that various modifications may be made without departing from the spirit and scope of the invention. Accordingly, other embodiments are within the scope of the following claims.

1. A method comprising:
surrounding a material with an energy-conducting medium;
irradiating the material; and
transferring heat energy away from the material through the energy-conducting medium at a rate sufficient to prevent radiation-induced damage to the material.

2. The method of claim 1, wherein the radiation comprises gamma radiation.

3. The method of claim 2, wherein the radiation is released at a controlled rate.

4. The method of claim 3, wherein the controlled rate is between 2 and 10 kilo-Gray/hour.

5. The method of claim 4, wherein the total energy absorbed by the material is between 10 and 40 kilo-Gray.

6. The method of claim 1, wherein the material comprises a human or animal tissue or a biological substance.

7. The method of claim 6, wherein the tissue comprises skin or tendon.

8. The method of claim 7, wherein the tissue is wetted.

9. The method of claim 1, wherein the energy-conducting medium comprises a physical support for the material.

10. The method of claim 1, wherein the energy-conducting medium conforms to the shape of the material.

11. The method of claim 1, wherein the energy-conducting medium comprises boron-nitride.

12. A method comprising:
sandwiching a material between layers of an energy-conducting medium having a higher conduction of energy flux in a lateral plane than in a normal plane;
placing the sandwiched material in a cold sink in thermal communication with the energy-conducting medium; and
applying radiation to the material in the normal plane.

13. A method comprising:
irradiating a material to inactivate microbes thereon;
conducting deleterious heat generated by the radiation through a heat-conducting medium; and
providing a cold sink for the draining of the conducted energy with which the heat-conducting medium is in thermal communication.

14. The method of claim 13, wherein the material comprises human or animal tissue, a biological substance or a synthesized polymeric.

15. An apparatus comprising:
a support having a first and a second surface;
an energy-conducting medium applied to at least the first and second surfaces of the support to conduct greater energy flux in a lateral plane than in a normal plane; and
a sealed sleeve to contain at least the support and the energy conducting medium.

16. The apparatus of claim 15, wherein the support comprises wetted foam.

17. The apparatus of claim 16, wherein the foam comprises hydrophilic polyurethane open-cell foam sheet.

18. The apparatus of claim 15, further comprising a cold sink external to the sleeve and in thermal communication with the energy-conducting medium.

19. The apparatus of claim 15, wherein the energy-conducting medium is boron-nitride.

20. The apparatus of claim 15, wherein the sleeve comprises a polypropylene bag.

21. A system comprising:
an irradiating device;
two or more cooling pads supporting material to be irradiated between the pads, the pads being in thermal communication with the material and capable of greater energy-conduction in a lateral plane than in a plane normal to the material; and
a cold sink in thermal communication with the cooling pads.

22. The system of claim 21, wherein each cooling pad comprises a wetted foam pad having an energy-conducting medium on one or more surfaces of the pad and sealed in a sleeve.
